(19) United States
(12) Patent Application Publication
(10) Pub. No.: US 2010/0092557 A1
(43) Pub. Date: Apr. 15, 2010

(54) Dosage Form Comprising Immediate Release Naproxen and Sustained Release Opioid Analgesic

(76) Inventors: Guy Vergnault, Loechle (FR); Pascal Grenier, Kappeien (FR)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/520,690

(22) PCT Filed: Dec. 11, 2007
(86) PCT No.: PCT/EP2007/010803

§ 371 Date: Nov. 17, 2009

(30) Foreign Application Priority Data
Dec. 21, 2006 (GB) 0625646.5

(51) Int. Cl. A61K 9/24; A61K 31/485; A61P 25/04

(52) U.S. Cl. 424/472; 514/282

(57) ABSTRACT

A dosage form adapted for twice-a-day administration comprising a naproxen species in an immediate release phase and an opioid analgesic in a sustained release phase. The dosage form is useful in the treatment of pain.

This invention relates to formulations containing a naproxen species and an opioid analgesic, to a method of making said formulations, and the use of the formulations in treating pain, in particular pain associated with inflammation such as post-operative pain, dental pain and arthritic pain.

Naproxen is a propionic acid derivative related to the group of non-steroidal anti-inflammatory drugs (NSAIDs). The chemical name for naproxen is (S)-6-methoxy-a-methyl-2-naphthaleneacetic acid. Naproxen is a pharmaceutically active species, although salts and derivatives of naproxen may be used in dosage forms. A commonly employed salt is the sodium salt—(S)-6-methoxy-a-methyl-2-naphthaleneacetic acid sodium salt. As used herein, the term naproxen species is used to describe naproxen and also any pharmaceutically acceptable salt or derivative of the acid, such as naproxen sodium salt.

Naproxen has a relatively long half-life (approximately 14 hours) compared with some of the other commonly used NSAIDs. For example, aspirin (0.25 hours), diclofenac (1.1 hours), flufenamic acid (1.4 hours), ibuprofen (2.1 hours), acetaminophen (4 hours), indomethacin (4.6 hours), ketoprofen (1.8 hours), and tiaprofenic acid (3.0 hours) have significantly shorter half lives. Yet it has a short half life when compared to others such as phenylbutazone (68 hours) and piroxicam (57 hours).

As will be described more fully below, the peculiar half life of naproxen makes it a particularly suitable ingredient when compared with practically all the other aforementioned NSAIDs, to employ in unit dosage forms of the present invention with an opioid analgesic in the treatment of pain, and particularly pain associated with inflammation.

It is known in the pharmaceutical arts to develop sustained release dosage forms containing opioid analgesics, which provide extended duration of effect when orally administered compared to conventional immediate release formulations that must be dosed 4 to 6 times daily, and twice-a-day and even once-a-day dosage forms of opioid analgesics have been described. It is also known to combine such opioid analgesics with NSAIDs in unit dosage form.

U.S. Pat. No. 5,968,551 describes a once-a-day sustained release dosage form containing an opioid analgesic. The dosage form may contain other drugs including NSAIDs. It is stated that the other drug or drugs may be in sustained release form or immediate release form. In the context of a once-a-day opioid sustained release formulation such as this, the use of naproxen in an immediate release layer would make no sense clinically because whereas the sustained release opioid is formulated to give 24 hour coverage, the naproxen in an immediate release phase would only give about 12 hours coverage and another naproxen dosage form would have to be taken to cover the additional 12 hours to prevent any breakthrough pain events associated with inflammation. It can easily be appreciated that this would considerably complicate a patient's dosage regimen.

EP649657 describes a treatment for arthritis using NSAID/opioid combinations. It is stated that dosage forms may contain both immediate release and sustained release phases. However, no mention is made as to which active agent should be provided in which phase. All of the examples disclose both actives agents in an immediate release phase.

EP220805 describes multi-layer tablets containing both NSAID and opioid analgesics. The NSAID and the opioid are physically separated into discrete layers. At least one layer must contain the NSAID and at least one layer must contain the opioid analgesic. The reason for the separation of the actives is explained in terms of avoiding perceived incompatibility between ingredients. The specification does state that the layers may be adapted for “normal” or sustained release. In the examples a naproxen/opioid formulation is described wherein both layers are adapated for “normal”, i.e. immediate release.

U.S. Pat. No. 5,908,848 discloses combinations of NSAIDs and opioid analgesics. The dosage forms may be formulated to give both immediate release and sustained release of the active ingredients. However, no mention is made as to which ingredient should be placed in which environment and all the examples describe formulations wherein both the active agents are provided in the same environment.

Finally, US2003/0092724 discloses formulations of NSAID/opioid analgesics. However, both actives are present in both immediate release and sustained release layers.

There remains a need to provide improved pain-management therapies that are effective in the treatment of pain, particularly pain associated with inflammation, that have a reduced frequency of dosing compared with conventional immediate release formulations for convenience of the patient, and which are relatively straightforward and inexpensive to produce.

The need is met by the present invention, which in a first aspect provides a unit dosage form suitable for twice-a-day administration to a patient in need of treatment comprising a first phase adapted for immediate release of a naproxen species, and a second phase adapted for the sustained release of opioid analgesic.

A dosage form that is “suitable for” twice-a-day administration should contain naproxen species and an opioid analgesic respectively in environments such that they are each released in a physiological medium to provide effective non-toxic plasma concentrations over a 12 hour period.

Unlike formulations described in the prior art, it is a characteristic of a dosage form of the present invention that all, or substantially all of the naproxen species is employed in the immediate release phase of the dosage form. This requirement follows from a consideration of the peculiar half-life of naproxen. An immediate release naproxen species will provide effective plasma concentrations of naproxen over a 12 hour period before the next dosage form in the twice-a-day schedule has to be taken.

Furthermore, it is far easier to formulate a naproxen species in a standard immediate release phase than it is to formulate it in a polymer matrix for sustained release. Immediate release formulations need do nothing more than to quickly distintegrate upon contact with a physiological medium to release the naproxen species.

Conversely, it is far more complicated to formulate an active agent in a sustained release phase. In sustained release formulations, the release-controlling matrix must remain intact over several hours in physiological media in order to release an active agent with the desired release rate over extended periods of time. This requires careful selection of certain qualities and quantities of release-controlling polymers and other excipients. It will be appreciated by the skilled person that the latitude of a formulator in its selection of said certain qualities and quantities of release-controlling polymers and excipients required to achieve a desired release profile can be influenced greatly by the nature and quantity of active agent that is to be employed in the sustained-release phase. In particular, if large amounts of active agent are to be employed, the formulator is limited in the amount of polymers and other excipients that can be used having regard to the fact that the overall size of the dosage form cannot be too large as to be unacceptable for administration to a patient orally.

In a dosage form according to the present invention if it is desired to elicit an effective anti-inflammatory response, relatively large amounts of naproxen species should be employed, making the use of an immediate release phase as the delivery vehicle for naproxen species particularly important. In order to deliver a preferred anti-inflammatory dose of naproxen to a patient, a dosage form must contain dosages of naproxen species that will provide to a patient 500 mg or more of naproxen, more particularly 500 to 750 mg of naproxen.

In a preferred embodiment of the invention, the naproxen species employed is the naproxen sodium salt, and the amount of sodium salt employed is such as to provide an effective anti-inflammatory dose of naproxen as aforementioned.

As stated hereinabove, it is preferred that all, or substantially all, of the naproxen species is contained in the immediate release phase. However, small amounts of naproxen may be contained in the sustained release phase. If employed in the sustained release phase the amount of naproxen species should preferably not exceed more than about 5% by weight of the total amount thereof, more particularly not exceed 1 to 3%, still more particularly less than 1% by weight, e.g. 0.01 to 1%.

The term “immediate release” as used in the present invention takes its art-recognised meaning. A phase is considered to act with immediate release if it meets disintegration and/or dissolution requirements for immediate release solid oral dosage forms as set out, for example in the United States Pharmacopoeia.

Preferably, an immediate release phase of a formulation according to the present invention will disintegrate within about 15 minutes in an aqueous medium. Disintegration does not imply complete dissolution of the ingredients contained in the phase. Complete disintegration is a state in which any residue of the phase remaining in test apparatus is a soft mass having no palpably firm core, as is more fully elaborated in the aforementioned Pharmacopoeia.

An apparatus for determining disintegration rates is defined in USP 26/NF21 under chapter 701. Therein an apparatus is described that consists of a basket-rack assembly, a 1000 ml beaker having an inside volume for receiving an immersion fluid, a thermostatic arrangement for heating said fluid between about 35 and 39 degrees centigrade, and a device for lowering the basket into the immersion fluid at a constant frequency of between about 29 and 32 cycles per minute. The apparatus is more fully described in the USP monograph mentioned above (which is hereby incorporated by reference) and needs no further elaboration here. Likewise, a procedure for carrying out the disintegration measurement is disclosed in the foregoing monograph, which is incorporated herein by reference.

The dissolution characteristics of the immediate release phase are preferably such that it displays about 50% dissolution within about 60 minutes in a buffered solution at a temperature of 37 degrees centigrade with a paddle speed of 100 rpm using paddle method apparatus no. 2. USP 26/NF 21 (“711 Dissolution”) describes compendial test methods and apparatus, which enables investigators to assess that the dissolution requirements are met, and this document is also incorporated by reference.

The term “sustained release” used in relation to the second phase means that that phase is adapted to release a drug substance within a certain time, or at a certain location to accomplish a therapeutic objective not possible using a conventional immediate release phase. More particularly, it means that the release of a drug substance is such that the blood plasma levels of the substance are maintained within a therapeutic range and below a toxic level for a period of about 12 hours.

As used herein, the term “opioid analgesic” is meant to describe a diverse group of drugs, of natural, synthetic, or semi-synthetic origin, that display opium or morphine-like properties. Opioid analgesics include, without limitation, morphine, heroin, hydromorphone, oxymorphone, buprenorphine, levorphanol, butorphanol, codeine, dihydrocodeine, hydrocodone, oxycodone, meperidine, methadone, nalbulphine, opium, pentazocine, propoxyphene, as well as less widely employed compounds such as alfentanil, allylprodine, alphaprodine, anileridine, benzylmorphine, bezitramide, clonitazene, cyclazocine, desomorphine, dextromoramide, dezocine, diampromide, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, hydroxypethidine, isomethadone, ketobemidone, levallorphan, levophenacylmorphan, lofentanil, meptazinol, metazocine, metopon, myrophine, narceine, nicomorphine, norpipanone, papvretum, phenadoxone, phenomorphan, phenazocine, phenoperidine, piminodine, propiram, sufentanil, tramadol, tilidine, and salts thereof.

A particularly preferred opioid analgesic useful in the present invention is hydrocodone, more particularly the bitartrate salt of hydrocodone.

The amount of opioid analgesic employed in the sustained release phase will depend on the nature of that opioid and the particular pain condition being treated. Generally, however, one might employ between 0.5 to 100 mg of opioid analgesic in a unit dosage form according to the present invention.

When the opioid analgesic is hydrocodone bitartrate it is preferred to use an amount of material of 5 to 60 mg, more particularly 5 to 30 mg, still more particularly 5 to 15 mg of hydrocodone bitartrate.

If it is desired to place an amount of the opioid analgesic in the immediate release phase in order to elicit a rapid onset of analgesia the present invention permits of this. When it is desired to include an opioid analgesic in the immediate release phase, it is preferred if about up to one third of the total dose is placed in the immediate release phase and two thirds in the sustained release phase.

The dosage forms of the present invention may take any convenient form. They may be provided as tablets, capsules, multi-particulates in sachet or capsule form or any other practical dosage form. Preferably however, the immediate release phase and the sustained release phase may be provided in the form of layers of a tablet. The layers may be formed concentrically or they may be formed contiguously in a sandwich-like fashion. In another alternative embodiment the immediate release phase and the sustained release phase may form discrete populations of powders or beads.

In a preferred aspect of the present invention the dosage form is provided in the form or a multilayered tablet wherein the layers are arranged in a sandwich-like fashion. There may be one or more immediate release and sustained release layers. The layers may be arranged one atop the other, or there may be employed support layers that contain no active agents interposed between immediate release and sustained release layers.

Tablet excipients are employed in the immediate release phase and the sustained release phase to enhance the bulk properties of the dosage form and to effect the desired release profiles. These excipients typically include diluents or fillers, which add bulk to a formulation to enable formulations of a desired size to be prepared; binders or adhesives, which promote the adhesion of the particles of a formulation to maintain the integrity of the dosage form; disintegrants or disintegrating agents, which promote the break-up of the dosage form after ingestion to make the ingredients more readily available; anti-adherents, glidants or lubricants, which enhance the flow of the tableting materials, for example into tablet dies, prevent sticking of the formulation to tablet-making machinery; and miscellaneous adjuvants such as colourants and flavourants.

Suitable diluents include pharmaceutically acceptable inert fillers such as microcrystalline cellulose, lactose, dibasic calcium phosphate, saccharides, and/or mixtures of any of the foregoing. Examples of diluents include microcrystalline cellulose such as Avicel ph112, Avicel pH101 and Avicel pH102; lactose such as lactose monohydrate, lactose anhydrous, and Pharmatose DCL 21; dibasic calcium phosphate such as Emcompress; mannitol; starch; sorbitol; fructose; sucrose; and glucose. Diluents are carefully selected to match the specific formulation with attention paid to the compression properties. The diluent is preferably used in an amount of 10% to 90% by weight, more particularly 50% by weight, of the immediate release layers.

Suitable lubricants and glidants, including agents that act on the flowability of the powder to be compressed are, for example, colloidal silicon dioxide such as Aerosil 200; talc; stearic acid, magnesium stearate, calcium stearate, sodium stearyl fumarate, polyethylene glycol and sodium lauryl sulphate. The lubricant is preferably used in an amount of 0.5 to 2% by weight, in particular 1% by weight, of the immediate release phase.

Suitable binders include polyethylene glycols such as PEG 6000; cetostearyl alcohol; cetyl alcohol; polyoxyethylene alkyl ethers; polyoxyethylene castor oil derivatives; polyoxyethylene sorbitan fatty acid esters; polyoxyethylene stearates; poloxamers; waxes, alginic acids and salts thereof; HPC; HPMC; methylcellulose; maltodextrin and dextrin; povidone; gums; starch and modified starches. The binder preferably may be used in an amount of 2 to 10% by weight, more particularly 5% by weight, of the immediate release phase.

Suitable disintegrants include sodium starch glycolate, such as Explotab™, crospovidone such as Kollidon CL, polyplasdone XL, sodium carboxymethylcellulose, sodium croscarmellose such as AcDiSol, and starch. The disintegrant preferably may be used in an amount of 2 to 10% by weight, more particularly 5% by weight, of the immediate release phase.

The sustained release phase may contain any of the aforementioned adjuvants referred to in relation to the immediate release phase in the amounts mentioned. However, in addition the sustained release phase should contain a release rate controlling agent.

The term “release rate controlling agent” includes any agent that controls the rate of release of an ingredient in terms of duration or location in order to give a therapeutic effect not possible with a conventional immediate release formulation, and includes hydrophilic polymers, hydrophobic polymers or mixtures thereof, or copolymers thereof, or mixtures of these polymers and copolymers.

Examples of release-rate controlling agents to be used in this invention include hydroxyalkylcellulose, such as hydroxypropylcellulose and hydroxypropylmethylcellulose; poly(ethylene)oxide; alkylcellulose such as ethycellulose and methylcellulose; carboxymethylcellulose; hydrophilic cellulose derivatives; polyethylene glycol; cellulose acetate; cellulose acetate butyrate; cellulose acetate phthalate; cellulose acetate trimellitate; polyvinylacetate phthalate; hydroxypropylmethylcellulose phthalate; hydroxypropylmethylcellulose acetate succinate; poly(alkyl methacrylate); and poly (vinyl acetate). Other suitable hydrophobic polymers include polymers or copolymers derived from acrylic or methacrylic acid esters, copolymers of acrylic and methacrylic acid esters, zein, waxes, shellac and hydrogenated vegetable oils.

The release-rate-controlling agent preferably includes a hydroxypropyl methylcellulose (HPMC), a hydroxypropyl cellulose (HPC), a poly(ethylene oxide), an ethylcellulose or a combination thereof, preferably present in an amount of 10 to 90% based on the weight of the sustained release phase.

Preferred types of HPMC for use in accordance with the invention are those sold under the trademark Methocel (Dow Chemical Co.). Suitable Methocels include the K grades such as Methocel K15M, Methocel K100M, Methocel K100 LV and Methocel K4M. Other suitable Methocels include the E, F and J grades.

As HPCs there can be those sold under the trademark Klucel (Hercules, Inc.) or equivalents. Suitable Klucels include Klucel LF, Klucel JF, Klucel GF, Klucel MF and Klucel HF.

As poly(ethylene oxide)s there may be mentioned those sold under the trademark Sentry Polyox (Union Carbide Corp.) or equivalents. Suitable Polyoxs include the Polyox WSR grades such as Polyox WSR Coagulant, Polyox WSR-301, Polyox WSR-303, Polyox WSR N-12K, Polyox WSR N-60K, Polyox WSR-1105, Polyox WSR-205 and Polyox WSR N-3000.

As ethylcelluloses for use in accordance with the invention there can be mentioned those sold under the trademark Ethocel (Dow Chemical Co.) or equivalents.

The hydroxypropylmethylcelluloses preferably have a viscosity (2 wt % solution at 20.degree. C.) of about 5 to 100,000 cps, preferably 4,000 to 100,000 cps. Especially suitable are Methocel K types or their equivalents. The hydroxypropylcelluloses used according to the invention preferably have a number average molecular weight of about 80,000 to 1,150,000, more preferably 80,000 to 600,000.

Poly(ethylene oxide) preferably have number average molecular weights of about 100,000 to 7,000,000, more preferably 900,000 to 7,000,000. Especially suitable is Polyox WSR Coagulant, which has a molecular weight of 5,000,000. The ethylcelluloses used according to the invention preferably have a viscosity of about 3 to 110 cps, more preferably 7 to 100 cps.

Dosage forms of the present invention may be coated with coating materials to achieve all manner of desired effects: For example coatings may be provided to achieve an aesthetic effect (e.g. an attractive colour or pleasant taste) or information effect, e.g. a coating may be coloured to act as a visual cue for a patient identification of the correct medicament. Coatings may also be over-written with information relating to the dosage, or they may elicit a functional effect such as a handling effect, e.g. a smooth coating for ease of swallowing, or a stability effect, e.g. a moisture or light barrier during storage.

In order to facilitate the preparation of dosage forms described above there is provided, in a further aspect of the present invention, a process for the preparation of a dosage form according to the present invention.

Dosages in the form of tablets may be made by compression methods by the application of high pressures to powders or granulates utilizing steel punches and dies. In this manner a wide variety of shapes, sizes and surface markings can be formed depending on the size and design of the punches and dies employed. On an industrial scale they may be produced using rotary presses suitable for producing multilayer tablets, e.g. a Layer-Press, Manesty, Liverpool, United Kingdom. Presses generally operate at pressures of about 1000 to about 5000 kg/cm².

Granulates may be made by dry or wet granulation methods.

Dry granulation (formed by slugging) involves the compaction of powders at high pressure into large tablet compacts. Granulates may also be formed by pressing/pushing powders between rollers of a chilsonator to form thin and dense ribbons. These compacts are then milled and screened to form granulates of the desired particle size.

Wet granulation is a technique widely employed in the art and comprises the steps of i) weighing and blending pharmaceutical ingredient and excipients; ii) preparing a damp mass from the ingredients and excipients; iii) screening the mass into pellets or granules; iv) drying the granulate; v) sizing the granulate by screening; vi) adding lubricant, preferably magnesium stearate, as appropriate and blending; and vii) tabletting by compression.

Wet granulation is a preferred method of forming granulates according to the present invention. However, given that naproxen sodium salt reacts exothermically in the presence of water, which can in turn lead to polymorphisms in the naproxen sodium salt, it is preferred that when this naproxen species is employed the wet granulation step to form the granulate for the immediate release phase is carried out at a temperature to avoid, or substantially avoid, such polymorphism. Preferably, the wet granulation process is carried out at a controlled temperature of about 20 to 25° C. Temperature control can be achieved by equipping the granulation apparatus with a suitable cool water jacket.

Should coating of the compositions be required, this can be achieved using conventional coating techniques such as spray coating, pan coating or air suspension coating techniques generally known in the art.

All of the techniques discussed above are described in detail in Ansel's Pharmaceutical Dosage Forms and Drug Delivery Systems, Chapter 7, Seventh Edition, 1999 (Lippincott Williams & Wilkins), which is herein incorporated by reference.

Dosage forms of the present invention are useful in the management of severe to moderate post-operative pain, pain associated with sprains, strains and fractures, dental pain and also in acute exacerbations of chronic pain. Dosage forms may also be useful in acutely painful conditions expected to last for at least a few days such as certain post-surgical cases, and accidents/trauma.

The appropriate dosage and the intervals been each dosage will depend upon the subject being treated and the type and severity of the condition for which the subject is being treated. Generally speaking however, the subject will receive orally the equivalent of between 800 to 1200 mg, e.g. 1000 mg naproxen per day per 70 kg body weight, and the equivalent of about 20 to 60 mg per day per 70 kg body weight of an opioid, e.g. hydrocodone bitartrate, delivered as two doses.

In a particularly preferred embodiment of the present invention the composition will be provided in a unit dosage form containing the equivalent of 500 mg naproxen free-acid and 15 mg hydrocodone bitartrate.

Preferred dosage forms of the present invention upon ingestion release a first pulse of hydrocodone to reach a therapeutic plasma concentration within a short period of time, to effect rapid pain relief. Thereafter a second pulse reaches maintains therapeutic plasma concentrations over a 12 hour time period thereby to extend the duration of pain relief compared to conventional narcotic combinations. The levels of naproxen reach a therapeutic level shortly after ingestion thereby to maintain an anti-inflammatory blood plasma level over a 12 hour period.

The invention will now be further illustrated with reference to the following Example.

### EXAMPLE 1

Tablets consisting of a sustained release layer containing hydrocodone bitartrate, and an immediate release phase containing naproxen sodium and hydrocodone bitartrate are formed according to the following procedure:

Preparation of a Granular Mass for an Immediate Release Phase

| | | |
|---|---|---|
| Naproxen Sodium | 550 | parts |
| Hydrocodone bitartrate | 5 | parts |
| Plasdone K29-32 | 27 | parts |
| AcDiSol | 40 | parts |
| Magnesium Stearate (C. Erba) | 6.8 | parts |
| Aerosil 200 | 3.1 | parts |
| Talc | 13.1 | parts |

The naproxen sodium, hydrocodone bitartrate, Plasdone and a portion of the AcDisol are mixed together in a high shear blender and wetted with a granulation liquid of purified water at a temperature of 20 to 25° C. The mixture is then dried to a pre-defined moisture level in a fluidized bed drier, sieved at 1 mm with an oscillatory mill before adding the remaining AcDisol, magnesium stearate and silica. Thereafter, the mixture is mixed in a tumble mixer. The granular mass obtained exhibits good flow and compacting properties.

Preparation of Granular Mass for the Sustained Release Phase

| | | |
|---|---|---|
| Hydrocodone bitartrate | 10 | parts |
| Methocel K100M | 128 | parts |
| Compritol 888ATO | 52 | parts |
| Lactose Pulvis H₂O | 59 | parts |
| Blanose 7H4XF | 70.00 | parts |
| PLASDONE K29-32 | 17 | parts |
| Magnesium stearate | 4 | parts |
| Aerosil 200 | 4 | parts |

Hydrocodone bitartrate, and excipients other than magnesium stearate and colloidal silica are blended together in a high shear mixer and then wetted with purified water as a granulation liquid. The mixture is dried in a fluidized bed dryer to a predefined moisture content, and then sieved through a 1.57 mm screen of a cone mill. The resultant granules are mixed with the magnesium stearate and colloidal silica in a diffusion-type blender. The granular mixture obtained is free-flowing and has good compaction properties.

Preparation of a Granular Mass for the Support Layer

| | |
|---|---|
| Methocel K4M | 80 parts |
| Lactose Pulvis H2O | 80 parts |
| Compritol 888 ATO | 27.00 parts |
| Plasdone K29-32 | 10.00 parts |
| Magnesium stearate | 02.00 parts |
| Aerosil 200 | 01.00 parts |

The excipients excluding magnesium stearate and silica are mixed together in a high shear blender and wetted with a granulation liquid of purified water at a temperature of 20 to 25° C. The mixture is dried in a fluidized bed drier to a pre-defined moisture level, then sieved through a 1.27 mm screen of a cone mill before adding the magnesium stearate and colloidal silica. Thereafter, the mixture is mixed in a tumble mixer. The granular mass obtained exhibits good flow and compacting properties

Formation of a Tablet

A Manesty Layer Press (LP39) is used for the manufacture of compressed tablets. As well known to those skilled in the art, the rotary press consists of several feeding stations to receive the granular layer blends. The press is equipped with elongate concave punches.

The granular materials are fed into hoppers, set to deliver a defined mass of granular material. The operating pressure is set to a pressure of 1500 kg/cm². Compressed tablets are formed having 3 contiguous layers containing naproxen sodium and hydrocodone bitartrate in the immediate release layer, the support layer and hydrocodone bitartrate in the sustained release layer.

1. A dosage form suitable for twice-a-day administration to a patient in need of treatment comprising a first phase adapted for immediate release of a naproxen species, and a second phase adapted for the sustained release of an opioid analgesic.

2. The dosage form according to claim 1, wherein the dose of naproxen species is an effective anti-inflammatory dose.

3. The dosage form according to claim 1, wherein the naproxen species is present in a dose of 500 mg to 750 mg.

4. The dosage form according to claim 1, wherein the naproxen species is naproxen free-acid or the sodium salt thereof.

5. The dosage form according to claim 1, wherein the entire dosage of naproxen species is in the immediate release phase.

6. The dosage form according to claim 1, wherein the opioid analgesic is selected from the group consisting of morphine, heroin, hydromorphone, oxymorphone, buprenorphine, levorphanol, butorphanol, codeine, dihydrocodeine, hydrocodone, oxycodone, meperidine, methadone, nalbulphine, opium, pentazocine, propoxyphene, alfentanil, allylprodine, alphaprodine, anileridine, benzylmorphine, bezitramide, clonitazene, cyclazocine, desomorphine, dextromoramide, dezocine, diampromide, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, hydroxypethidine, isomethadone, ketobemidone, levallorphan, levophenacylmorphan, lofentanil, meptazinol, metazocine, metopon, myrophine, narceine, nicomorphine, norpipanone, papvretum, phenadoxone, phenomorphan, phenazocine, phenoperidine, piminodine, propiram, sufentanil, tramadol, tilidine, and salts thereof.

7. The dosage form according to claim 1 wherein the opioid analgesic is hydrocodone bitartrate.

8. The dosage form according to claim 7, wherein the hydrocodone bitartrate is present in an amount of 15 mg.

9. The dosage form according to claim 7, wherein a portion of the dose of the hydrocodone bitartrate is contained in the immediate release phase and a portion is contained in the sustained release phase.

10. The dosage form according to claim 7, wherein the naproxen species is present in an amount to provide to a patient 500 mg of naproxen free-acid.

11. The dosage form according to claim 1, in the form of a tablet and wherein the phases are provided as layers arranged in a sandwich-like manner.

12. A pharmaceutical package comprising a plurality of dosage forms as defined in any of the preceding claims together with instructions for the dosage forms to be administered twice-a-day.

13. The use of dosage forms of claim 1 in the treatment of pain.

14. The use according to claim 13 wherein the pain is post-operative pain, dental pain or pain associated with arthritis.
